(19) United States
(12) Patent Application Publication
(10) Pub. No.: US 2025/0017775 A1
(43) Pub. Date: Jan. 16, 2025

(54) TOOLS FOR MICROSURGICAL PROCEDURES

(71) Applicant: ForSight Robotics Ltd., Yokneam Illit (IL)

(72) Inventors: Ariel Gil, Haifa (IL); Yoav Golan, Tel Aviv (IL); Tal Korman, Tel Aviv (IL); Ofer Arnold, Ma'ale Zvia (IL); Daniel Glozman, Kfar Yona (IL); Zev Sohn, Karnei Shomron (IL)

(21) Appl. No.: 18/714,024

(22) PCT Filed: Dec. 1, 2022
(86) PCT No.: PCT/IB2022/061633

§ 371 Date: May 28, 2024

Related U.S. Application Data
(60) Provisional application No. 63/285,147, filed on Dec. 2, 2021.

(51) Int. Cl. A61F 9/007; A61B 17/00; A61B 17/29; A61B 17/3203; A61B 34/00; A61B 34/30; A61B 90/90

(52) U.S. Cl. CPC A61F 9/00754; A61B 2017/00323; A61B 2017/2932; A61B 17/3203; A61B 2034/301; A61B 34/72; A61B 2034/742; A61B 90/90

(57) ABSTRACT

Apparatus and methods are described for performing robotic microsurgery on a patient's body. Two or more tools (21) each include a mount-engagement portion (32) that defines a front recess (82) and a rear recess (84). A tool mount (34) defines a tool-receiving socket (86) securely holds the one or more tools. The tool-mount (34) includes a rear set of rollers (94) that are configured to be placed within the rear recess (84) and a front set of rollers (92) that are configured to be placed within the front recess (82). A tool-securement cover that is hingedly coupled to the tool-receiving socket (86) is configured to secure the tool (21) within the tool-receiving socket (86). A motor (93) rolls the tool (21) with respect to the tool mount (34), while the tool (21) is securely held within the tool mount (34). Other applications are also described.

### CROSS-REFERENCES TO RELATED APPLICATIONS

The present application claims priority from U.S. Provisional Patent Application No. 63/285,147 to Gil, filed Dec. 2, 2021, entitled “Tools for microsurgical procedures”, which is incorporated herein by reference.

### FIELD OF EMBODIMENTS OF THE INVENTION

Some applications of the present invention generally relate to medical apparatus and methods. Specifically, some applications of the present invention relate to apparatus and methods for performing microsurgical procedures in a robotic manner.

### BACKGROUND

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and their face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more incisions (and typically two or three incisions) are made in the cornea of the eye. The incision(s) are typically made using a specialized blade, which is called a keratome blade. At this stage, lidocaine is typically injected into the anterior chamber of the eye, in order to further anesthetize the eye. Following this step, a viscoelastic injection is applied via the corneal incision(s). The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incision, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. Further typically, a separate tool is used to perform suction during the phacoemulsification. When the phacoemulsification is complete, the remaining lens cortex (i.e., the outer layer of the lens) material is aspirated from the capsule. During the phacoemulsification and the aspiration, aspirated fluids are typically replaced with irrigation of a balanced salt solution, in order to maintain fluid pressure in the anterior chamber. In some cases, if deemed to be necessary, then the capsule is polished. Subsequently, the intraocular lens (IOL) is inserted into the capsule. The IOL is typically foldable and is inserted in a folded configuration, before unfolding inside the capsule. At this stage, the viscoelastic is removed, typically using the suction device that was previously used to aspirate fluids from the capsule. If necessary, the incision(s) is sealed by elevating the pressure inside the bulbus oculi (i.e., the globe of the eye), causing the internal tissue to be pressed against the external tissue of the incision, such as to force closed the incision.

### SUMMARY

In accordance with some applications of the present invention, a robotic system is configured for use in a microsurgical procedure, such as intraocular surgery. Typically, when used for intraocular surgery, the robotic system includes one or more robotic units (which are configured to hold tools), in addition to an imaging system, one or more displays, and a control-component unit (for example a control-component unit that includes a pair of control components, such as joysticks), via which one or more operators (e.g., healthcare professionals, such as a physician and/or a nurse) are able to control robotic units. Typically, the robotic system includes one or more computer processors, via which components of the system and the operators operatively interact with each other. The scope of the present application includes mounting one or more robotic units in any of a variety of different positions with respect to each other.

For some applications, a set of tools is provided, each of which includes a universal mount-engagement portion for engaging a tool mount of an end effector of the robotic unit, in accordance with some applications of the present invention. For some applications, the set of tools comprises a universal tool kit for use with the robotic unit that includes all tools that are typically used in a cataract procedure, a different ophthalmic procedure, and/or a different microsurgical procedure. For example, the set of tools typically includes one or more of the following tools: a keratome blade, an eye fixator, a paracentesis knife, a dispersive ophthalmic viscosurgical device (OVD) syringe, a cohesive ophthalmic viscosurgical device (OVD) syringe, a staining syringe (e.g., for staining the anterior lens with a stain such as trypan blue ophthalmic solution), a lidocaine syringe, forceps, a hydrodissection syringe, a phacoemulsification probe, a chopper, an irrigation/aspiration probe, an intraocular lens injector, an antibiotics syringe, and/or a Limbal Relaxing Incision (LRI) knife. For some applications, each of the tools includes one or more markers, which may be used to identify the tools and/or to determine the position and/or orientation of the tool.

Typically, the mount-engagement portion of the tools comprises a sleeve that is disposed around the outside of each of the tools. The sleeve includes a gear wheel, as well as a front recess and a rear recess. For some applications, at least one of the front and rear recesses has a frustoconical shape. Typically, the tool mount includes a socket for receiving the tool, as well as a tool-securement cover that is hingedly coupled to the tool-receiving socket and that is configured to secure the tool within the tool-receiving socket. Typically, in order to place the tool in the tool mount, the tool-securement cover is opened. The tool is then placed within the socket, before the tool-securement cover is closed such as to secure the tool within the tool-receiving socket.

For some applications, the tool is configured to be actuated to perform a function via a linear tool-actuation arm, which is disposed on the end effector and is configured to push a portion of the tool axially. For example, the linear tool-actuation arm may be configured to push the plunger of a syringe axially in a forward direction. For some applications, a portion of the tool is configured to be moved with respect to the patient's eye by the linear tool-actuation arm pushing a portion of the tool axially. As described hereinabove, typically, each of a plurality of different types of tools having different functions from each other includes universal mount-engagement portion. Thus, each of the tools is couplable with respect to the tool mount, in a manner that permits the tool to be rolled with respect to the tool mount. For some applications, an additional feature that facilitates use of the robotic unit with each of the plurality of tools is that the linear tool-actuation arm is configured to automatically slide and/or fold in order to accommodate a larger tool (such as a phacoemulsification probe).

For some applications, a further feature that facilitates use of the robotic unit with each of the plurality of tools is that many of the tools are actuated to perform their respective functions using the linear tool-actuation arm. For some applications, in order for one or more of the tools to be actuated using the linear tool-actuation arm, the tool includes a motion-conversion mechanism for converting the linear motion (which is applied to a portion of the tool via the linear tool-actuation arm) to a different mechanical motion such as to actuate the tool.

Typically, movement of the robotic units (and/or control of other aspects of the robotic system) is at least partially controlled by the one or more operators. For example, the operator may receive images of the patient's eye and the robotic units, and/or tools disposed therein, via a display. Based on the received images, the operator typically performs steps of the procedure. For some applications, the operator provides commands to the robotic units via a control-component unit. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

Typically, the control-component unit includes one or more joysticks that are configured to correspond to respective robotic units of the robotic system. For example, the system may include first and second robotic units, and the control-component unit may include first and second joysticks to be operated by the operators right and left hands. For some applications, the control-component joysticks comprise respective control-component tools therein (in order to replicate the robotic units). Typically, the computer processor determines the XYZ location and orientation of a tip of the control-component tool, and drives the robotic unit such that the tip of the actual tool that is being used to perform the procedure tracks the movements of the tip of the control-component tool.

For some applications, the joystick includes an actuation mechanism. Typically, the actuation mechanism is disposed toward a tip of the control-component tool such that the operator can actuate the actuation mechanism without requiring movement of the operator's hand after moving the control-component tool. Further typically, the actuation mechanism is actuated by the operator performing a squeezing action. For example, the actuation mechanism may be a button, or a pressure sensitive pad. For some applications, the computer processor receives an input that is indicative of a tool that is coupled to the end effector. For example, the operator may input an indication of the tool into the computer processor. Alternatively or additionally, each of the tools may have a tool-identification component (e.g., a marker), and the computer processor may be configured to automatically derive which tool is currently coupled to the end effector by identifying the tool-identification component within an image of the tool. Further alternatively or additionally, the computer processor may be configured to automatically derive which tool is currently coupled to the end effector by analyzing an image of the tool even without using the tool-identification component.

Typically, in response to the operator actuating the actuation mechanism, the computer processor operates one or more actuation components of the robotic unit in a manner that is such as to actuate the tool that is coupled to the end effector to perform its function. For example, in response to detecting that a syringe of a certain type is currently coupled to the end effector, the computer processor may drive the linear tool-actuation arm to advance the plunger of the syringe through a given distance. Or, in response to detecting that the keratome blade is currently coupled to the end effector, the computer processor may drive the keratome blade to move in such a manner as to make an incision in the anterior capsule of the patient's eye.

There is therefore provided, in accordance with some applications of the present invention, apparatus for performing robotic microsurgery on a portion of a body of a patient, the apparatus including:
two or more tools each of which includes a mount-engagement portion that defines a front recess and a rear recess;
a tool mount configured to securely hold the one or more tools, the tool mount defining a tool-receiving socket configured to receive the tool, and including:
   a rear set of rollers that are configured to be placed within the rear recess of the mount-engagement portion;
   a front set of rollers that are configured to be placed within the front recess of the mount-engagement portion; and
   a tool-securement cover that is hingedly coupled to the tool-receiving socket and that is configured to secure the tool within the tool-receiving socket, at least a portion of the rear set of rollers and at least a portion of the front set of rollers being disposed on the tool-securement cover; and
   one or more motors associated with the tool mount and configured to roll the tool with respect to the tool mount, while the tool is securely held within the tool mount.

In some applications, the mount-engagement portion includes a first gear wheel and the tool mount includes a second gear wheel that is configured to be rolled by the one or more motors, and the mount-engagement portion is sized such that when the tool is secured within the tool-receiving socket, the first gear wheel is positioned such as to engage the second gear wheel.

In some applications, the front recess has a frustoconical shape, and the front set of rollers are configured to be disposed at an angle with respect to an axis of the tool, when the tool is securely held within the tool mount, such as to conform to the shape of the frustoconical recess.

In some applications, the rear recess has a frustoconical shape, and the rear set of rollers are configured to be disposed at an angle with respect to an axis of the tool, when the tool is securely held within the tool mount, such as to conform to the shape of the frustoconical recess.

In some applications, the tool mount is configured such that insertion of the front rollers into the front recess and the rear rollers into the rear recess is such as to allow the tool to roll with respect to the tool mount while securely holding the tool in place with respect to the tool mount both radially and axially.

In some applications, the tool mount is configured such that insertion of the front rollers into the front recess and the rear rollers into the rear recess is such that the front rollers and rear rollers act as radial bearings during rolling of the tool.

In some applications, the mount-engagement portion includes a sleeve that is disposed around the outside of each of the tools.

There is further provided, in accordance with some applications of the present invention, apparatus for performing robotic microsurgery on a portion of a body of a patient, the apparatus including:
a plurality of tools having different functions from each other, each of the tools defining a mount-engagement portion that has a common shape;
an end effector that includes a tool mount that is configured to securely hold each of the plurality of tools by engaging with the mount-engagement portion of each of the tools;
the end effector including a linear tool-actuation arm that is configured to actuate the tools by moving linearly,
at least one of the tools including a motion-conversion mechanism for converting the linear motion to a different mechanical motion such as to actuate the tool.

In some applications, the linear tool-actuation arm is configured to automatically move in response to being retracted to a given distance from tool mount in order to accommodate a larger tool.

In some applications, the linear tool-actuation arm includes a spring mechanism, and is configured to fold automatically in response to being retracted to a given distance from tool mount, by means of the spring mechanism being activated.

In some applications, at least one of the tools includes forceps that includes tips, and a motion-conversion mechanism for converting the linear motion to transverse motion of the tips toward each other, such as to close the tips.

In some applications, the motion-conversion mechanism includes a hinged sleeve that is disposed around the proximal ends of the tips and a ramped surface that is not parallel to an axis of forceps, configured such that as the hinged sleeve is advanced past the ramped surface, the hinged sleeve is configured to be pushed transversely inwards, to thereby cause the tips to close.

In some applications, the motion-conversion mechanism includes a ramped surface and rollers disposed around proximal portions of the tips, configured such that as the rollers advance past the ramped surface, the rollers are pushed transversely inwards, to thereby cause the tips to close.

In some applications, at least one of the tools includes a tool having a steerable tip, and a motion-conversion mechanism for converting the linear motion to non-linear motion of the steerable tip.

In some applications:
the steerable tip is hinged,
the motion-conversion mechanism includes a pusher and a steering wire, and
linear motion of the pusher is conveyed to the steering wire thereby causing the hinged tip to bend.

There is further provided, in accordance with some applications of the present invention, apparatus for performing a procedure on a portion of a body of a patient, the apparatus including:
a plurality of tools having different functions from each other;
a robotic unit including an end effector that is couplable to each of the plurality of tools and that is configured to move each of the plurality of tools;
the end effector including one or more actuation components that are configured to actuate the plurality of tools to perform their respective functions;
a control joystick that is configured to be moved by an operator such as to cause the end effector to move a tool that is coupled to the end effector in a corresponding manner,
the control joystick including an actuation mechanism disposed toward a tip of the joystick such that the operator can actuate the actuation mechanism without requiring movement of a hand of the operator after moving the joystick with the hand; and
a computer processor configured to:
   receive an input that is indicative of a tool that is coupled to the end effector; and
   in response to the operator actuating the actuation mechanism, to control the one or more actuation components in a manner that is such as to actuate the tool that is coupled to the end effector to perform its function.

In some applications, the control joystick includes a control-component tool and the tip of the joystick includes the tip of the control-component tool, and the computer processor is configured to determine an XYZ location and orientation of the tip of the control-component tool and to drive the end effector to move a tip of the tool that is coupled to the end effector in a corresponding manner.

In some applications, the actuation mechanism is configured to be actuated by the operator performing a squeezing action.

In some applications, the computer processor is configured to receive the input that is indicative of the tool that is coupled to the end effector by analyzing an image of the tool.

In some applications, each of the tools includes a tool-identification component, and the computer processor is configured to receive the input that is indicative of the tool that is coupled to the end effector by identifying the tool-identification component within the image of the tool.

There is further provided, in accordance with some applications of the present invention, apparatus for injecting a plurality of different substances into a portion of a body of a patient, the apparatus including:
a syringe including a cannula and a barrel;
a robotic unit including an end effector that is couplable to the syringe;
a plurality of lumens, each of the lumens being fluidically coupled to a respective one of the substances and being reversibly fluidically couplable to the barrel of the syringe,
the robotic unit being configured to:
receive an input that a given one of the substances should be injected into the portion of the patient's body, and, in response thereto:
   fluidically couple the lumen that is fluidically coupled to the given substance to the barrel of the syringe, and
   inject the given substance from the barrel into the portion of the patient's body via the cannula.

In some applications, the syringe includes a mechanical plunger, and the robotic unit is configured to inject the given substance from the barrel into the portion of the patient's body via the cannula, by advancing the mechanical plunger though the barrel.

In some applications, the syringe includes a plunger selected from the group consisting of: a pneumatic plunger, and a hydraulic plunger, and the robotic unit is configured to inject the given substance from the barrel into the portion of the patient's body by activating the selected plunger.

In some applications, the plurality of lumens are arranged in a manifold arrangement along a length of the barrel.

In some applications, the syringe includes a fluid-selection plunger, and the robotic unit is configured to move the fluid-selection plunger such as to place respective lumens in fluid communication with the barrel.

In some applications, the plurality of lumens are arranged in parallel with each other, with each of the lumens leading to the barrel.

In some applications, the syringe includes a revolving chamber, and the robotic unit is configured to rotate the revolving chamber such that respective lumens are placed into fluid communication with the barrel depending on a rotational position of the revolving chamber.

There is further provided, in accordance with some applications of the present invention, a method for injecting a plurality of different substances into a portion of a body of a patient, the method including:
coupling an end effector of a robotic unit to a syringe that includes a cannula, a barrel, and a plurality of lumens, each of the lumens being fluidically coupled to a respective one of the substances and being reversibly fluidically couplable to the barrel of the syringe;
providing an input to the robotic unit that a given one of the substances should be injected into the portion of the patient's body, to thereby drive the robotic unit to fluidically couple the lumen that is fluidically coupled to the given substance to the barrel of the syringe; and driving the robotic unit to inject the given substance from the barrel into the portion of the patient's body via the cannula.

There is further provided, in accordance with some applications of the present invention, apparatus for performing robotic microsurgery on a portion of a body of a patient, the apparatus including:
forceps that include tips and a mount-engagement portion;
an end effector that includes a tool mount that is configured to securely hold the forceps by engaging with the mount-engagement portion of the forceps;
the end effector including a linear tool-actuation arm that is configured to push a portion of the forceps distally with respect to the mount-engagement portion of the forceps,
the forceps including a motion-conversion mechanism configured to convert the distal motion of the portion of the forceps to transverse motion of the tips toward each other, such as to close the tips.

In some applications, the motion-conversion mechanism includes a hinged sleeve that is disposed around the proximal ends of the tips and a ramped surface that is not parallel to an axis of forceps, configured such that as the hinged sleeve is advanced past the ramped surface, the hinged sleeve is configured to be pushed transversely inwards, to thereby cause the tips to close.

In some applications, the motion-conversion mechanism includes a ramped surface and rollers disposed around proximal portions of the tips, configured such that as the rollers advance past the ramped surface, the rollers are pushed transversely inwards, to thereby cause the tips to close.

In some applications, the linear tool-actuation arm is configured to automatically move in response to being retracted to a given distance from tool mount in order to accommodate a larger tool.

In some applications, the linear tool-actuation arm includes a spring mechanism, and is configured to fold automatically in response to being retracted to a given distance from tool mount, by means of the spring mechanism being activated.

In some applications, the forceps include a button, the linear tool-actuation arm is configured to push the button distally, and the motion-conversion mechanism includes a hinged joint that is configured to cause the tips of the forceps to be closed by distal ends of the forceps arms pivoting toward each other.

In some applications, the forceps include forceps arms and the hinged joint includes joint arms and a central portion, and the hinged joint is configured such that the pushing of the button pushes the central portion of the hinged joint linearly, thereby causing the joint arms to push the proximal ends of the forceps arms such that the proximal ends of the forceps arms pivot outwardly with respect to each other, thereby causing the tips of the forceps to be closed by distal ends of the forceps arms pivoting toward each other.

There is further provided, in accordance with some applications of the present invention, a method for performing robotic microsurgery on a portion of a body of a patient, the method including:
placing forceps into a tool mount of an end effector, such that the tool mount securely holds the forceps by engaging with the mount-engagement portion of the forceps;
activating a linear tool-actuation arm of the end effector to push a portion of the forceps distally with respect to the mount-engagement portion of the forceps,
such that a motion-conversion mechanism of the forceps converts the distal motion of the portion of the forceps to transverse motion of tips of the forceps toward each other, such as to close the tips.

There is further provided, in accordance with some applications of the present invention, apparatus for performing robotic microsurgery on a portion of a body of a patient, the apparatus including:
a tool comprising a steerable tip;
a robotic unit comprising an end effector that is couplable to the tool and that is configured to move the tool;
the end effector comprising a linear-actuation arm that is configured to drive the steerable tip to move non-linearly, by applying linear motion to a portion of the tool.

In some applications:
the steerable tip is hinged; and
the tool comprises a pusher and a steering wire, and linear motion of the pusher is conveyed to the steering wire thereby causing the hinged tip to bend.

The present invention will be more fully understood from the following detailed description of embodiments thereof, taken together with the drawings, in which:

### BRIEF DESCRIPTION OF THE DRAWINGS

FIG. 1 is a schematic illustration of a robotic system that is configured for use in a microsurgical procedure, such as intraocular surgery, in accordance with some applications of the present invention;

FIG. 2 is a schematic illustration of a set of tools each of which includes a universal mount-engagement portion for engaging a tool mount of an end effector of a robotic unit, in accordance with some applications of the present invention;

FIGS. 3A-C are schematic illustrations of respective views of universal mount-engagement portion of tools, in accordance with some applications of the present invention;

FIGS. 4A and 4B are schematic illustrations of respective views of a tool mount in an open state, in accordance with some applications of the present invention;

FIGS. 4C and 4D are schematic illustrations of a tool disposed within the tool mount, and with the tool mount respectively in an open state and in a closed state, in accordance with some applications of the present invention;

FIGS. 5A and 5B are schematic illustrations of a syringe for use with the robotic system, the syringe being configured to inject a plurality of different substances into a portion of the subject's body, in accordance with some applications of the present invention;

FIGS. 6A and 6B are schematic illustrations of forceps for use with the robotic system, in accordance with some applications of the present invention;

FIGS. 7A and 7B are schematic illustrations of forceps for use with the robotic system, in accordance with some alternative applications of the present invention;

FIGS. 8A and 8B are schematic illustrations of forceps for use with the robotic system, in accordance with some further alternative applications of the present invention;

FIG. 9 is a schematic illustration of a tool having a steerable tip for use with the robotic system, in accordance with some applications of the present invention;

FIG. 10 is a schematic illustration of a joystick for use with the robotic system, in accordance with some applications of the present invention.

### DETAILED DESCRIPTION OF EMBODIMENTS

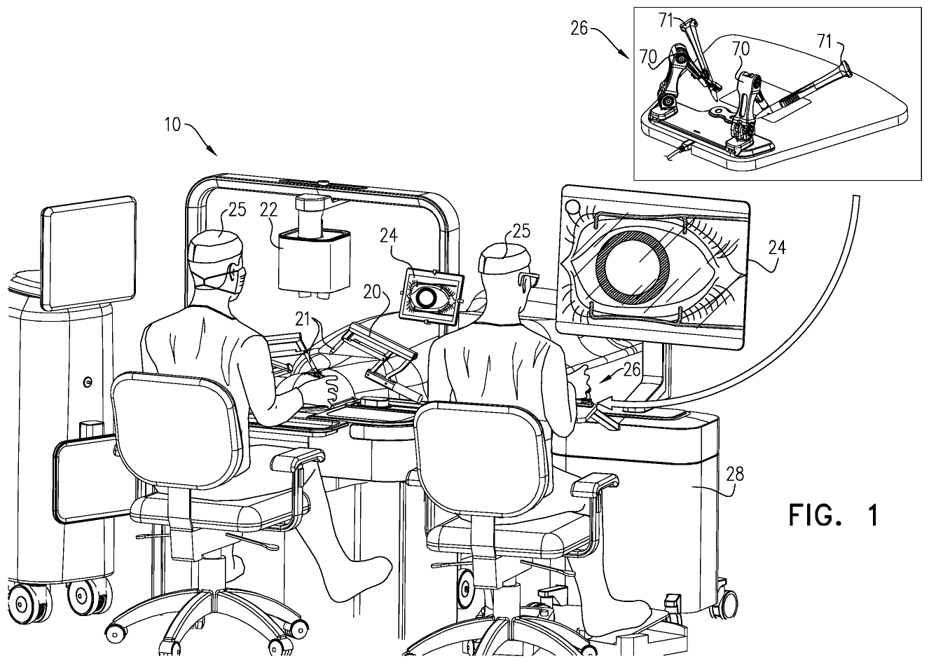

Reference is now made to FIG. 1, which is a schematic illustration of a robotic system 10 that is configured for use in a microsurgical procedure, such as intraocular surgery, in accordance with some applications of the present invention. Typically, when used for intraocular surgery, robotic system 10 includes one or more robotic units 20 (which are configured to hold tools 21), in addition to an imaging system 22, one or more displays 24 and a control-component unit 26 (for example a control-component unit that includes a pair of control components, such as joysticks 70, as shown in the enlarged portion of FIG. 1), via which one or more operators 25 (e.g., healthcare professionals, such as a physician and/or a nurse) are able to control robotic units 20. Typically, robotic system 10 includes one or more computer processors 28, via which components of the system and operator(s) 25 operatively interact with each other. The scope of the present application includes mounting one or more robotic units in any of a variety of different positions with respect to each other.

Typically, movement of the robotic units (and/or control of other aspects of the robotic system) is at least partially controlled by one or more operators 25 (e.g., healthcare professionals, such as a physician and/or a nurse). For example, the operator may receive images of the patient's eye and the robotic units, and/or tools disposed therein, via display 24. Typically, such images are acquired by imaging system 22. Typically, the imaging system includes one or more cameras and/or one or more microscopes. For some applications, imaging system 22 is a stereoscopic imaging device and display 24 is a stereoscopic display. Based on the received images, the operator typically performs steps of the procedure. For some applications, the operator provides commands to the robotic units via control-component unit 26. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

Typically, the control-component unit includes one or more joysticks 70 that are configured to correspond to respective robotic units 20 of the robotic system. For example, as shown, the system may include first and second robotic units, and the control-component unit may include first and second joysticks, as shown. For some applications, the control-component joysticks comprise respective control-component tools 71 therein (in order to replicate the robotic units), as shown in FIG. 1. Typically, the computer processor determines the XYZ location and orientation of the tip of the control-component tool 71, and drives the robotic unit such that the tip of the actual tool 21 that is being used to perform the procedure tracks the movements of the tip of the control-component tool.

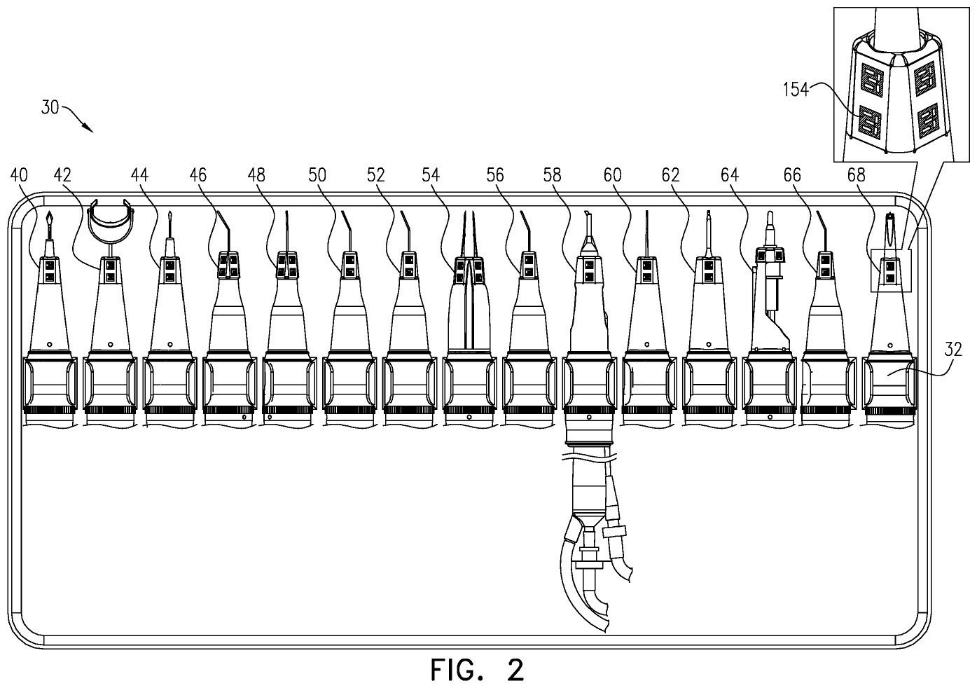

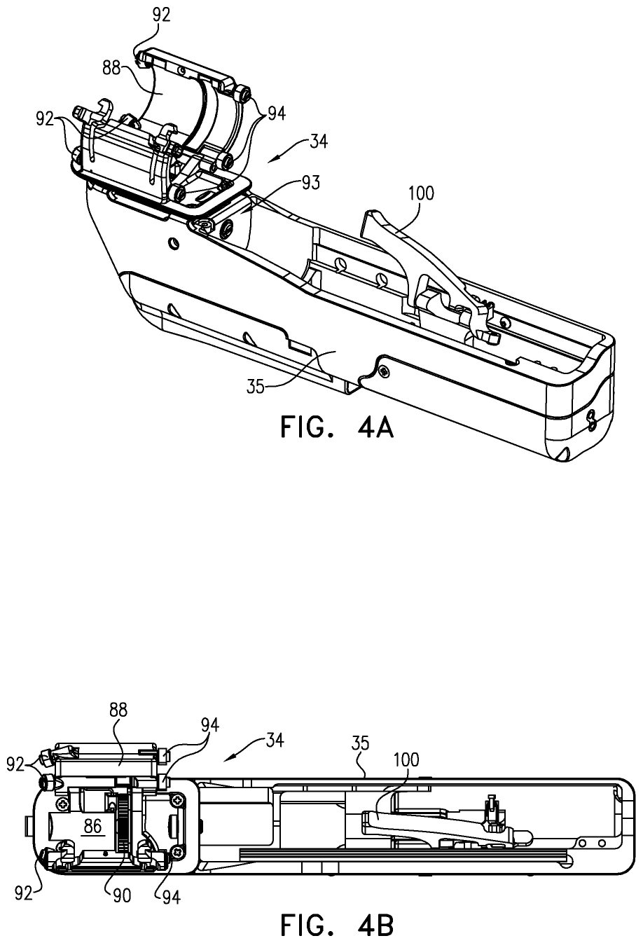

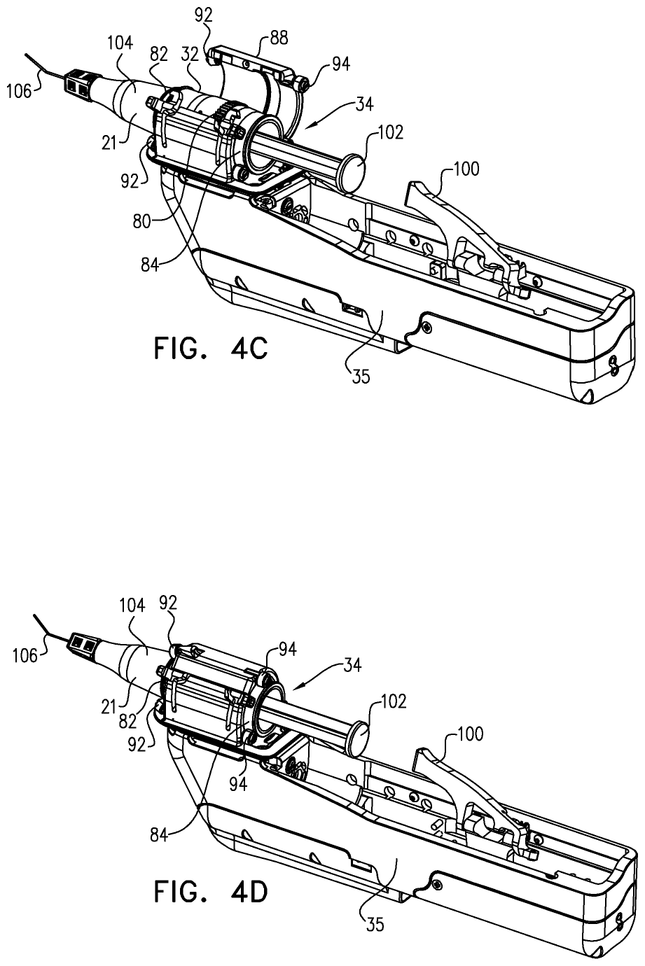

Reference is now made to FIG. 2, which is a schematic illustration of a set 30 of tools each of which includes a universal mount-engagement portion 32 for engaging a tool mount 34 of an end effector 35 (tool mount and end effector being shown in FIGS. 4A-D) of robotic unit 20, in accordance with some applications of the present invention. For some applications, set 30 of tools 21 comprises a universal tool kit for use with the robotic unit 20 that includes all tools that are typically used in a cataract procedure, a different ophthalmic procedure, and/or a different microsurgical procedure. For example, as shown in FIG. 2, the set of tools typically includes one or more of the following tools: a keratome blade 40, an eye fixator 42, a paracentesis knife 44, a dispersive ophthalmic viscosurgical device (OVD) syringe 46, a cohesive ophthalmic viscosurgical device (OVD) syringe 48, a staining syringe 50 (e.g., for staining the anterior lens with a stain such as trypan blue ophthalmic solution), a lidocaine syringe 52, forceps 54, a hydrodissection syringe 56, a phacoemulsification probe 58, a chopper 60, an irrigation/aspiration probe 62, an intraocular lens injector 64, an antibiotics syringe 66, and/or a Limbal Relaxing Incision (LRI) knife 68. For some applications, each of the tools includes one or more markers 154, which may be used to identify the tools and/or to determine the position and/or orientation of the tool. Some functions of markers 154 are described in further detail hereinbelow.

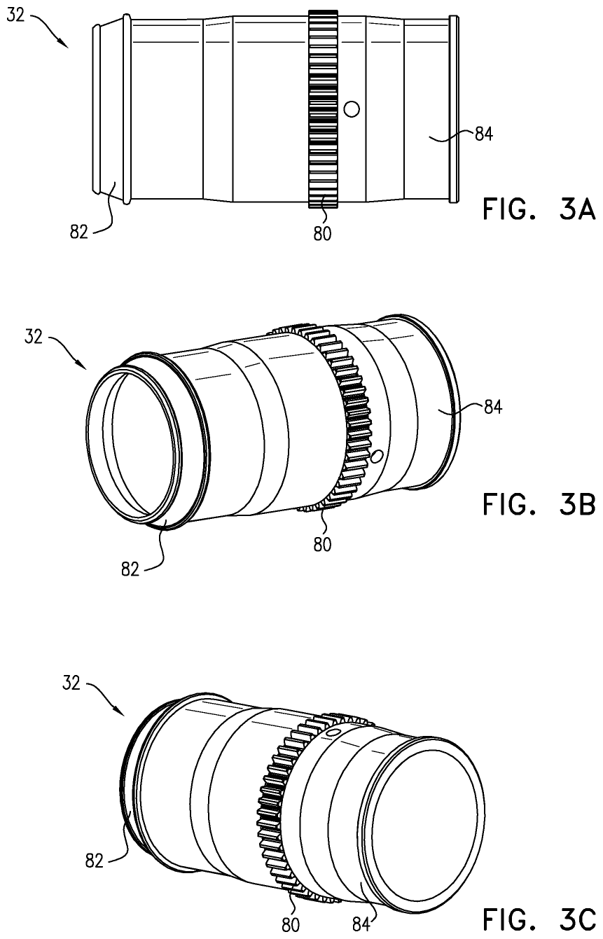

Reference is now made to FIGS. 3A-C, which are schematic illustrations of respective views of universal mount-engagement portion 32 of tools 21, in accordance with some applications of the present invention. Reference is also made to FIGS. 4A and 4B, which are schematic illustrations of respective views of tool mount 34 of end effector 35 in an open state, as well as to FIGS. 4C and 4D, which are schematic illustrations of a tool 21 disposed within the tool mount, with the tool mount respectively in an open state and in a closed state, in accordance with some applications of the present invention. Tool mount 34 is typically coupled to, or formed integrally with, an end effector 35 of the robotic unit.

Typically, mount-engagement portion 32 of tools 21 comprises a sleeve that is disposed around the outside of each of the tools. The sleeve includes a gear wheel 80, as well as a front recess 82 and a rear recess 84. For some applications, at least one of the front and rear recesses has a frustoconical shape. For example, as shown, front recess 82 has a frustoconical shape. Typically, tool mount 34 includes a socket 86 (shown in FIG. 4B) for receiving the tool, as well as a tool-securement cover 88 that is hingedly coupled to the tool-receiving socket and that is configured to secure the tool within the tool-receiving socket. Typically, in order to place the tool in the tool mount, the tool-securement cover is opened (as shown in FIGS. 4A-B). The tool is then placed within socket 86 (as shown in FIG. 4C), before the tool-securement cover 88 is closed such as to secure the tool within the tool-receiving socket (as shown in FIG. 4D).

Typically, mount-engagement portion 32 is sized such that when the tool is secured within the tool-receiving socket (as described in further detail hereinbelow), gear wheel 80 of the mount-engagement portion is positioned such as to engage a gear wheel 90 of the tool mount. A motor 93 of robotic unit 20 is typically configured to drive the tool to roll with respect to the tool mount by driving gear wheel 90 to roll gear wheel 80, to thereby cause the tool to roll. For some applications, the tool is secured within the tool-receiving socket by front rollers 92 being placed within front recess 82 and rear rollers 94 being placed within rear recess 84. (It is noted that in order to place the rollers into the recesses, the tool may be moved with respect to the rollers as an alternative to, or in addition to, the rollers being moved with respect to the tool.) As noted above, for some applications, at least one of the front and rear recesses has a frustoconical shape. Typically, for such applications, the rollers that are configured to be placed within the frustoconical recess are disposed at an angle with respect to the axis of the tool. For example, as shown, front recess 82 has a frustoconical shape, and front rollers 92 are disposed at an angle with respect to the axis of the tool, such as to conform to the shape of the frustoconical recess.

As noted above, robotic unit 20 is typically configured to drive the tool to roll with respect to the tool mount by driving gear wheel 90 to roll gear wheel 80, to thereby cause the tool to roll. Typically, the insertion of the front rollers 92 into front recess 82 and rear rollers 94 into rear recess 84 is such that the rollers act as radial bearings during rolling of the tool. Further typically, the insertion of the front rollers 92 into front recess 82 and rear rollers 94 into rear recess 84 is such as to allow the tool to roll with respect to the tool mount while securely holding the tool in place with respect to the tool mount both radially and axially.

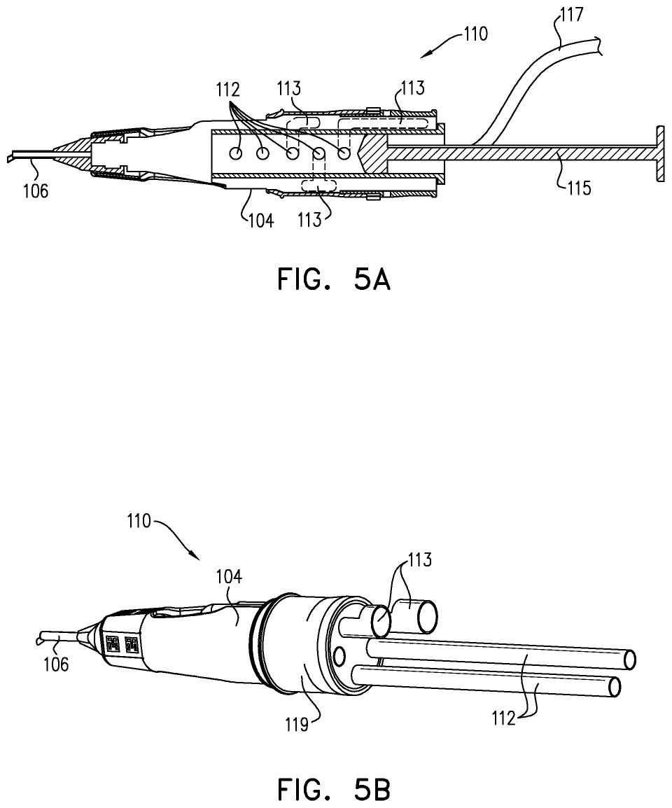

For some applications, tool 21 is configured to be actuated to perform a function via a linear tool-actuation arm 100, which is disposed on end effector 35 and is configured to push a portion of the tool axially. Typically, the linear tool-actuation arm pushes the portion of the tool distally with respect to the tool mount and the mount-engagement portion (i.e., such that the portion of the tool distally moves distally relative to the tool mount and the mount-engagement portion. For example, as shown in FIG. 4C-D, the tool that is placed within tool mount is a syringe (e.g., dispersive ophthalmic viscosurgical device (OVD) syringe 46, cohesive ophthalmic viscosurgical device (OVD) syringe 48, staining syringe 50, lidocaine syringe 52, hydrodissection syringe 56, intraocular lens injector 64, and/or antibiotics syringe 66). Typically, the syringe includes a plunger 102, a barrel 104, and a cannula 106 (barrel and cannula shown in FIGS. 5A-B, for example). In such cases, the linear tool-actuation arm is configured to push plunger 102 of the syringe axially in a forward direction. For some applications, a portion of the tool is configured to be moved with respect to the patient's eye by linear tool-actuation arm 100 pushing a portion of the tool axially. As described hereinabove, for some applications, front recess 82 has a frustoconical shape, and front rollers 92 are disposed at an angle with respect to the axis of the tool. Typically, by being configured in this manner, the front rollers are configured to counter forces that would otherwise push the entire tool axially forward relative to the tool mount. For example, when the linear tool-actuation arm pushes the plunger of the syringe axially in the forward direction, the front roller typically applies a counterforce to the syringe to prevent barrel 104 of the syringe from being pushed axially forward relative to the tool mount. More generally, a recess having a frustoconical shape typically provides additional axial stability to the tool relative to if the recess were to be coaxial with the axis of the tool.

Reference is now made to FIGS. 5A and 5B, which are schematic illustrations of a syringe 110 for use with the robotic system, the syringe being configured to inject a plurality of different substances into a portion of the subject's body, in accordance with some applications of the present invention. As described hereinabove (with reference to FIG. 2), typically, a plurality of different syringes are used in an ophthalmic procedure, such as a cataract procedure. Dispersive ophthalmic viscosurgical device (OVD) syringe 46, cohesive ophthalmic viscosurgical device (OVD) syringe 48, staining syringe 50 (e.g., for staining the anterior lens with a stain such as trypan blue ophthalmic solution), lidocaine syringe 52, hydrodissection syringe 56, intraocular lens injector 64, and antibiotics syringe 66, are all examples of syringes that are typically used. For some applications, a multipurpose syringe that is configured to inject a plurality of different substances is used.

As described hereinabove, typically, the syringe includes barrel 104, and cannula 106. Typically, the syringe is mounted on tool mount 34, such that the syringe is coupled to end effector 35. For some applications, syringe 110 includes a plurality of lumens 112, each of the lumens being fluidically coupled to a respective one of the substances that is to be injected, and each of the lumens being reversibly fluidically couplable to barrel 104 of the syringe.

For some application, lumens 112 are arranged in a manifold arrangement along the length of barrel 104, for example, as shown in FIG. 5A. The other end of each of the lumens is typically fluidically coupled to a respective one of the substances that is to be injected. For some applications, at least some of the lumens are fluidically coupled to capsules 113 containing respective substances. The capsules are optionally disposed alongside barrel 104, as shown. For some applications, the syringe includes a fluid-selection plunger 115, which is configured to be moved such as to place respective lumens in fluid communication with the barrel. As described hereinabove, typically syringe 110 includes a plunger, which is typically a mechanical plunger configured to inject the selected substance into the subject. Alternatively, for some applications, the syringe includes a pneumatic plunger 117 as shown, which is configured to inject the selected fluid into the subject using pneumatic pressure. Alternatively or additionally, the syringe includes a different sort of plunger, e.g., a hydraulic plunger.

For some applications, lumens 112 are arranged in parallel with each other, with each of the lumens leading to the barrel, for example, as shown in FIG. 5B. The other end of each of the lumens is typically fluidically coupled to a respective one of the substances that is to be injected. For some applications, at least some of the lumens are fluidically coupled to capsules 113 containing respective substances. For some applications, respective lumens are placed into fluid communication with barrel 104 by rotating a revolving chamber 119, which is configured such that respective lumens are placed into fluid communication with barrel 104 depending on the rotational position of the revolving chamber. For some applications, the revolving chamber is rotated through respective rotational positions using a ratchet mechanism (not shown).

Typically, the robotic unit receives an input that a given one of the substances should be injected into the portion of the patient's body. For example, operator 25 may provide an input to computer processor 28 indicating that a given substance is to be injected (operator 25 and computer processor 28 being shown in FIG. 1). In response thereto, the robotic unit fluidically couples the lumen that is fluidically coupled to the given substance to the barrel of the syringe (e.g., using one of the mechanisms described hereinabove). Subsequently, the robotic unit injects the given substance from the barrel into the patient's eye via the cannula by advancing the plunger through the barrel (and/or using a different type of plunger, e.g., a pneumatic and/or a hydraulic plunger). For example, the computer processor may drive linear tool-actuation arm 100 to advance the plunger through the barrel.

As described hereinabove, typically, each of a plurality of different types of tools 21 having different functions from each other includes universal mount-engagement portion 32. Thus, each of the tools is couplable with respect to tool mount 34, in a manner that permits the tool to be rolled with respect to the tool mount. For some applications, an additional feature that facilitates use of robotic unit 20 with each of the plurality of tools is that linear tool-actuation arm 100 is configured to automatically slide and/or fold in order to accommodate a larger tool (such as phacoemulsification probe 58). For some applications, the tool-actuation arm is configured to fold automatically in response to being retracted to a given distance from tool mount 34. In this manner, the tool-actuation arm may be folded automatically such as to accommodate the insertion of a larger tool, such as a phacoemulsification probe, into the tool mount, without requiring removal and/or manual folding of the tool-actuation arm. Typically, the tool-actuation arm is configured to fold automatically by means of a spring mechanism being activated. Further typically, in response to the tool-actuation arm being moved closer to the tool mount, the tool-actuation arm is configured to automatically unfold (e.g., via a spring mechanism being activated). For some applications, rather than being configured to fold automatically, the arm is configured to be moved in a different manner such as to accommodate the insertion of a larger tool, such as a phacoemulsification probe, into the tool mount, without requiring removal and/or manual movement of the tool-actuation arm. For example, the arm may be configured to be automatically retracted, e.g., using an electromechanical actuator, a spring mechanism, etc.

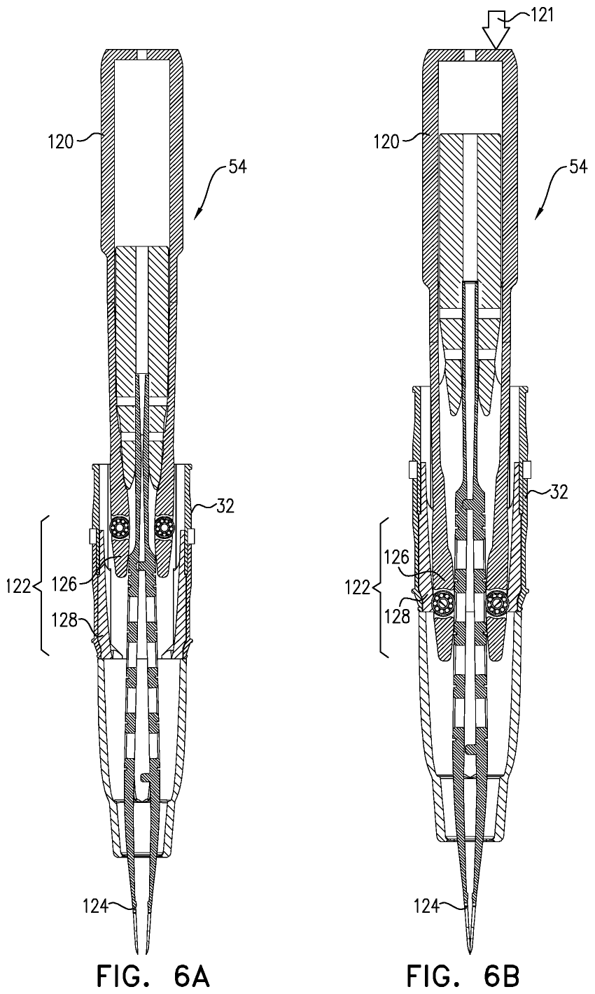

For some applications, a further feature that facilitates use of robotic unit 20 with each of the plurality of tools is that many of the tools are actuated to perform their respective functions using linear tool-actuation arm 100. For some applications, in order for one or more of the tools to be actuated via using linear tool-actuation arm 100, the tool includes a motion-conversion mechanism for converting the linear motion (which is applied to a portion of the tool via linear tool-actuation arm 100) to a different mechanical motion such as to actuate the tool. Examples of this are provided with reference to FIGS. 6A-B, 7A-B, 8A-B and 9.

Reference is now made to FIGS. 6A and 6B, which are schematic illustrations of forceps 54 for use with the robotic system, in accordance with some applications of the present invention. For some application, the forceps are configured such that in response to linear motion being applied to a handle 120 of the forceps via linear tool-actuation arm 100 (as indicated by arrow 121 and the transition from FIG. 6A to 6B), a motion-conversion mechanism 122 causes tips 124 of the forceps to close against each other by moving transversely. For some applications, the motion-conversion mechanism comprises a hinged sleeve 126 that is disposed around the proximal ends of the tip and a ramped surface 128 (e.g., a curved surface or an angled surface) that is not parallel to the axis of forceps. As the hinged sleeve is advanced past the surface 128, the hinged sleeve is pushed transversely inwards, which causes the tips to close.

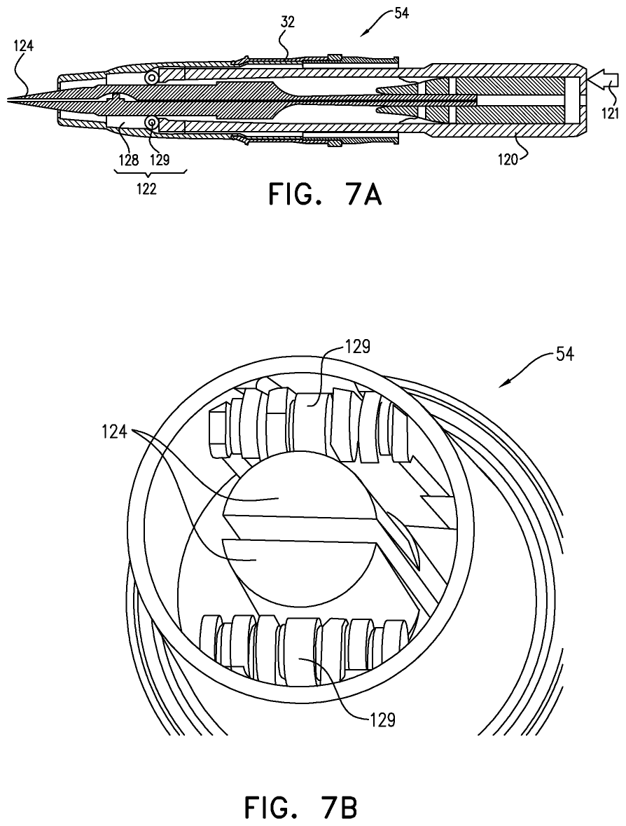

Reference is now made to FIGS. 7A and 7B, which are schematic illustrations of forceps 54 for use with the robotic system, in accordance with some alternative applications of the present invention. As described with reference to forceps 54 as shown in FIGS. 6A-B, the forceps are configured such that in response to linear motion being applied to a handle 120 of the forceps via linear tool-actuation arm 100 (as indicated by arrow 121), a motion-conversion mechanism 122 causes tips 124 of the forceps to close against each other by moving transversely. For some applications, the motion-conversion mechanism comprises ramped surface 128 (e.g., a curved surface or an angled surface) and rollers 129 disposed around a proximal portion of the tips. As the rollers advance past the ramped surfaces, the rollers are pushed transversely inwards, which causes the tips to close.

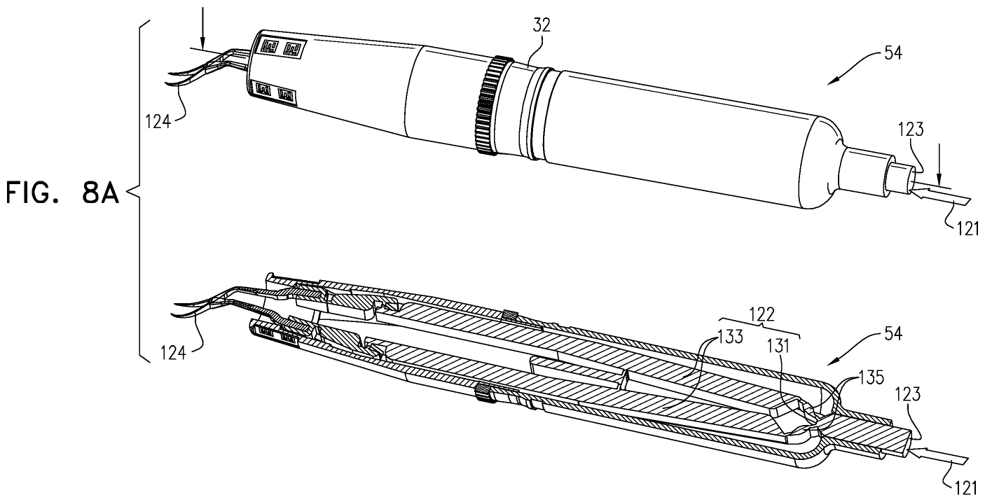

Reference is now made to FIGS. 8A and 8B, which are schematic illustrations of forceps 54 for use with the robotic system, in accordance with some alternative applications of the present invention. The forceps are configured such that in response to linear motion being applied to a button 123 of the forceps via linear tool-actuation arm 100 (as indicated by arrow 121), a motion-conversion mechanism 122 causes two forceps arms 133 to pivot about a hinged joint 131. The tips of the forceps are coupled to the ends of respective forceps arms. For some applications, the hinged joint is configured such that the pushing of button 123 pushes a central portion of the hinged joint linearly, which causes arms 135 of the hinged joint to push the proximal ends of forceps arms 133, such that forceps arms 133 pivot outwardly with respect to each other. In turn, this causes the tips of the forceps to be closed by the distal ends of the forceps arms pivoting toward each other, as shown in the transition from FIG. 8A to FIG. 8B.

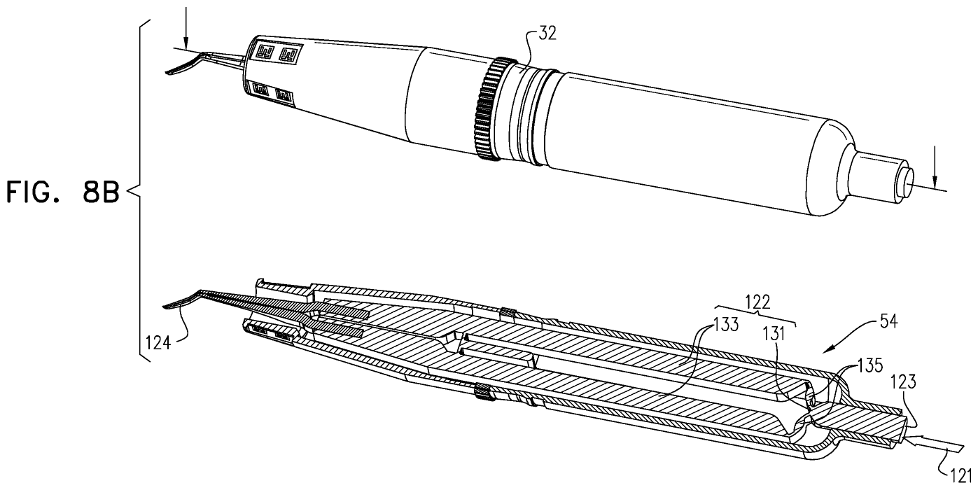

Reference is now made to FIG. 9, which is a schematic illustration of a tool 130 having a steerable tip for use with robotic system 10, in accordance with some applications of the present invention. For some applications, tool 130 includes a steerable tip 132. Typically, linear tool-actuation arm 100 (shown in FIGS. 4A-D) is configured to drive the steerable tip to move non-linearly, by applying linear motion to a portion of the tool. For example, the tool may include a pusher 134 which is coupled to a motion-conversion mechanism that is configured to convert linear motion of the pusher to non-linear motion of the steerable tip. For some applications, the tip is hinged and the tip is coupled to a steering wire 138 (e.g., a nitinol steering wire). For some applications, the pusher is coupled to the steering wire via a rack-and-pinion mechanism 142 (and optionally one or more reduction gears 144). Linear movement of the pusher is conveyed to the steering wire via the rack-and-pinion mechanism 142 (and optionally reduction gears 144), which causes the hinged tip to bend.

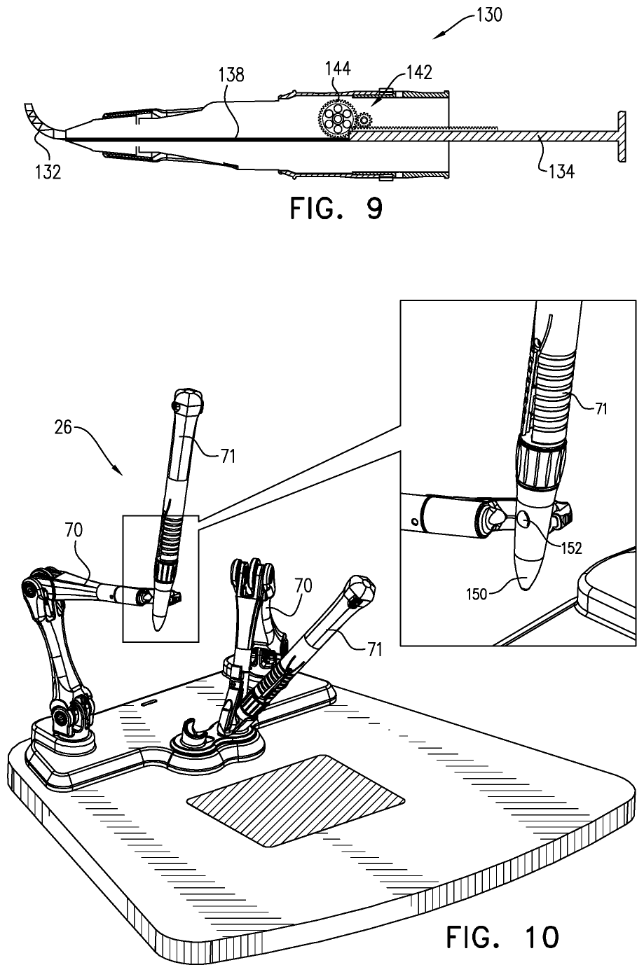

Reference is now made to FIG. 10, which is a schematic illustration of a control joystick for use with the robotic system, in accordance with some applications of the present invention. As described hereinabove with reference to FIG. 1, typically, movement of the robotic units (and/or control of other aspects of the robotic system) is at least partially controlled by one or more operators 25 (e.g., healthcare professional, such as a physician and/or a nurse). For example, the operator may receive images of the patient's eye and the robotic units, and/or tools disposed therein, via display 24. Based on the received images, the operator typically performs steps of the procedure. For some applications, the operator provides commands to the robotic units via control-component unit 26. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

Typically, the control-component unit includes one or more joysticks 70 that are configured to correspond to respective robotic units 20 of the robotic system. For example, as shown the system may include first and second robotic units, and the control-component unit may include first and second joysticks to be operated by the operators right and left hands, as shown. For some applications, the control-component joysticks comprise respective control-component tools 71 therein (in order to replicate the robotic units), as shown in FIG. 1. Typically, the computer processor determines the XYZ location and orientation of a tip 150 of the control-component tool 71, and drives the robotic unit such that the tip of the actual tool 21 that is being used to perform the procedure tracks the movements of the tip of the control-component tool.

For some applications, joystick 70 includes an actuation mechanism 152. Typically, the actuation mechanism is disposed toward a tip of the control-component tool 71 such that the operator can actuate the actuation mechanism without requiring movement of the operator's hand after moving the control-component tool 71. Further typically, the actuation mechanism is actuated by the operator performing a squeezing action. For example, the actuation mechanism may be a button, or a pressure sensitive pad. For some applications, computer processor 28 receives an input that is indicative of a tool that is coupled to the end effector. For example, the operator may input an indication of the tool into the computer processor. Alternatively or additionally, each of the tools may have a tool-identification component (e.g., marker 154 (shown in FIG. 2), and the computer processor is configured to automatically derive which tool is currently coupled to the end effector by identifying the tool-identification component within an image of the tool. Further alternatively or additionally, the computer processor is configured to automatically derive which tool is currently coupled to the end effector by analyzing an image of the tool even without using the tool-identification component.

Typically, in response to the operator actuating the actuation mechanism, the computer processor operates one or more actuation components of robotic unit 20 in a manner that is such as to actuate the tool that is coupled to the end effector to perform its function. For example, in response to detecting that a syringe of a certain type is currently coupled to the end effector, the computer processor may drive the linear tool-actuation arm to advance the plunger of the syringe through a given distance. Or, in response to detecting that the keratome blade is currently coupled to the end effector, the computer processor may drive the keratome blade to move in such a manner as to make an incision in the anterior capsule of the patient's eye.

Although some applications of the present invention are described with reference to cataract surgery, the scope of the present application includes applying the apparatus and methods described herein to other medical procedures, mutatis mutandis. In particular, the apparatus and methods described herein to other medical procedures may be applied to other microsurgical procedures, such as general surgery, orthopedic surgery, gynecological surgery, otolaryngology, neurosurgery, oral and maxillofacial surgery, plastic surgery, podiatric surgery, vascular surgery, and/or pediatric surgery that is performed using microsurgical techniques. For some such applications, the imaging system includes one or more microscopic imaging units.

It is noted that the scope of the present application includes applying the apparatus and methods described herein to intraocular procedures, other than cataract surgery, mutatis mutandis. Such procedures may include collagen crosslinking, endothelial keratoplasty (e.g., DSEK, DMEK, and/or PDEK), DSO (descemet stripping without transplantation), laser assisted keratoplasty, keratoplasty, LASIK/PRK, SMILE, pterygium, ocular surface cancer treatment, secondary IOL placement (sutured, transconjunctival, etc.), iris repair, IOL reposition, IOL exchange, superficial keratectomy, Minimally Invasive Glaucoma Surgery (MIGS), limbal stem cell transplantation, astigmatic keratotomy, Limbal Relaxing Incisions (LRI), amniotic membrane transplantation (AMT), glaucoma surgery (e.g., trabs, tubes, minimally invasive glaucoma surgery), automated lamellar keratoplasty (ALK), anterior vitrectomy, and/or pars plana anterior vitrectomy.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described with reference to the Figures, computer processor 28 typically acts as a special purpose robotic-system computer processor. Typically, the operations described herein that are performed by computer processor 28 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for performing robotic microsurgery on a portion of a body of a patient, the apparatus comprising:
two or more tools each of which comprises a mount-engagement portion that defines a front recess and a rear recess:
a tool mount configured to securely hold the one or more tools, the tool mount defining a tool-receiving socket configured to receive the tool, and comprising:
a rear set of rollers that are configured to be placed within the rear recess of the mount-engagement portion:
a front set of rollers that are configured to be placed within the front recess of the mount-engagement portion; and
a tool-securement cover that is hingedly coupled to the tool-receiving socket and that is configured to secure the tool within the tool-receiving socket, wherein at least a portion of the rear set of rollers and at least a portion of the front set of rollers are disposed on the tool-securement cover; and
one or more motors associated with the tool mount and configured to roll the tool with respect to the tool mount, while the tool is securely held within the tool mount.

2. The apparatus according to claim 1, wherein the mount-engagement portion comprises a first gear wheel and the tool mount comprises a second gear wheel that is configured to be rolled by the one or more motors, and wherein the mount-engagement portion is sized such that when the tool is secured within the tool-receiving socket, the first gear wheel is positioned such as to engage the second gear wheel.

3. The apparatus according to claim 1, wherein the front recess has a frustoconical shape, and wherein the front set of rollers are configured to be disposed at an angle with respect to an axis of the tool, when the tool is securely held within the tool mount, such as to conform to the shape of the frustoconical recess.

4. The apparatus according to claim 1, wherein the rear recess has a frustoconical shape, and wherein the rear set of rollers are configured to be disposed at an angle with respect to an axis of the tool, when the tool is securely held within the tool mount, such as to conform to the shape of the frustoconical recess.

5. The apparatus according to claim 1, wherein the tool mount is configured such that insertion of the front rollers into the front recess and the rear rollers into the rear recess is such as to allow the tool to roll with respect to the tool mount while securely holding the tool in place with respect to the tool mount both radially and axially.

6. The apparatus according to claim 1, wherein the tool mount is configured such that insertion of the front rollers into the front recess and the rear rollers into the rear recess is such that the front rollers and rear rollers act as radial bearings during rolling of the tool.

7. The apparatus according to claim 1, wherein the mount-engagement portion comprises a sleeve that is disposed around the outside of each of the tools.

8. Apparatus for performing robotic microsurgery on a portion of a body of a patient, the apparatus comprising:
a plurality of tools having different functions from each other, each of the tools defining a mount-engagement portion that has a common shape:
an end effector that comprises a tool mount that is configured to securely hold each of the plurality of tools by engaging with the mount-engagement portion of each of the tools:
the end effector comprising a linear tool-actuation arm that is configured to actuate the tools by moving linearly,
wherein at least one of the tools comprises a motion-conversion mechanism for converting the linear motion to a different mechanical motion such as to actuate the tool.

9. The apparatus according to claim 8, wherein the linear tool-actuation arm is configured to automatically move in response to being retracted to a given distance from tool mount in order to accommodate a larger tool.

10. The apparatus according to claim 9, wherein the linear tool-actuation arm comprises a spring mechanism, and is configured to fold automatically in response to being retracted to a given distance from tool mount, by means of the spring mechanism being activated.

11. The apparatus according to claim 8, wherein at least one of the tools comprises forceps that comprises tips, and a motion-conversion mechanism for converting the linear motion to transverse motion of the tips toward each other, such as to close the tips.

12. The apparatus according to claim 11, wherein the motion-conversion mechanism comprises a hinged sleeve that is disposed around the proximal ends of the tips and a ramped surface that is not parallel to an axis of forceps, configured such that as the hinged sleeve is advanced past the ramped surface, the hinged sleeve is configured to be pushed transversely inwards, to thereby cause the tips to close.

13. The apparatus according to claim 11, wherein the motion-conversion mechanism comprises a ramped surface and rollers disposed around proximal portions of the tips, configured such that as the rollers advance past the ramped surface, the rollers are pushed transversely inwards, to thereby cause the tips to close.

14. The apparatus according to claim 8, wherein at least one of the tools comprises a tool having a steerable tip, and a motion-conversion mechanism for converting the linear motion to non-linear motion of the steerable tip.

15. The apparatus according to claim 14, wherein:
the steerable tip is hinged,
the motion-conversion mechanism comprises a pusher and a steering wire, and
linear motion of the pusher is conveyed to the steering wire thereby causing the hinged tip to bend.

16. Apparatus for performing a procedure on a portion of a body of a patient, the apparatus comprising:
a plurality of tools having different functions from each other:
a robotic unit comprising an end effector that is couplable to each of the plurality of tools and that is configured to move each of the plurality of tools;
the end effector comprising one or more actuation components that are configured to actuate the plurality of tools to perform their respective functions;
a control joystick that is configured to be moved by an operator such as to cause the end effector to move a tool that is coupled to the end effector in a corresponding manner,
the control joystick comprising an actuation mechanism disposed toward a tip of the joystick such that the operator can actuate the actuation mechanism without requiring movement of a hand of the operator after moving the joystick with the hand; and
a computer processor configured to:
receive an input that is indicative of a tool that is coupled to the end effector; and
in response to the operator actuating the actuation mechanism, to control the one or more actuation components in a manner that is such as to actuate the tool that is coupled to the end effector to perform its function.

17. The apparatus according to claim 16, wherein the control joystick comprises a control-component tool and the tip of the joystick comprises the tip of the control-component tool, and wherein the computer processor is configured to determine an XYZ location and orientation of the tip of the control-component tool and to drive the end effector to move a tip of the tool that is coupled to the end effector in a corresponding manner.

18. The apparatus according to claim 16, wherein the actuation mechanism is configured to be actuated by the operator performing a squeezing action.

19. The apparatus according to claim 16, wherein the computer processor is configured to receive the input that is indicative of the tool that is coupled to the end effector by analyzing an image of the tool.

20. The apparatus according to claim 19, wherein each of the tools comprises a tool-identification component, and wherein the computer processor is configured to receive the input that is indicative of the tool that is coupled to the end effector by identifying the tool-identification component within the image of the tool.

21-36. (canceled)
